(19) United States
(12) Patent Application Publication
(10) Pub. No.: US 2008/0242696 A1
(43) Pub. Date: Oct. 2, 2008

(54) CRYSTALLINE GRANISETRON BASE AND PRODUCTION PROCESS THEREFOR

(75) Inventors: Yael Gafni, Rosh Ha'ayin (IL); Alex Weisman, Kiryat Ekron (IL); Itai Adin, Beer Sheva (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: CHEMAGIS LTD., Bnei Brak (IL)

(21) Appl. No.: 11/691,778

(22) Filed: Mar. 27, 2007

(51) Int. Cl. A61K 31/439; A61P 1/08; C07D 401/02

(52) U.S. Cl. 514/304; 546/126

(57) ABSTRACT

Provided is crystalline granisetron base form I and processes for producing crystalline granisetron base form I, which is suitable for preparing, e.g., granisetron salts such as, e.g., the hydrochloride salt. Also provided is a process for producing a salt of granisetron from crystalline granisetron base form I.

### BACKGROUND OF THE INVENTION

Granisetron hydrochloride is an anti-emetic drug, used for treatment or prophylaxis of emesis and post operative nausea and vomiting. Granisetron hydrochloride is marketed under the trade name Kytril as solution for injection as well as tablets. The chemical name of granisetron is N-(endo-9-methyl-9-azabicyclo[3.3.2]non-3-yl)-1-methylindazole-3-carboxamide and it is represented by the following structural formula (I):

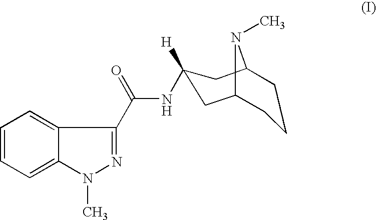

Granisetron is usually administered as the hydrochloride salt for relieving the symptoms of vomiting and nausea in cancer patients. Recently the U.S. Food and Drug Administration (FDA) has accepted an investigational New Drug (IND) application for transdermal granisetron patch, Sancuso™, for the prevention of chemotherapy-induced nausea and vomiting (CINV). The Sancuso™ Phase III study is now underway in Europe and in the U.S. Typically, a non-oral form such as transdermal patch uses granisetron base as the active ingredient.

The preparation of granisetron base is described in U.S. Pat. No. 6,268,498 without referring to the solid state characteristics of granisetron. The preparation of granisetron base is further described in example 3 of U.S. Pat. No. 7,071,209 (hereinafter the '209 patent), having a melting point of 121-122° C. The '209 patent is silent with regard to the solid state of granisetron base as well as to the solid state of the hydrochloride salt, although the infrared spectrum of granisetron base is mentioned.

Since granisetron base is used as the active pharmaceutical ingredient in transdermal dosage forms as described above, there is a need in the art for a stable, well-defined crystalline granisetron base polymorph, which may be conveniently used as the active pharmaceutical ingredient in the preparation of non-oral forms comprising granisetron base, and simple processes for preparing such a polymorph. The present invention provides such a polymorph and processes.

### SUMMARY OF THE INVENTION

The present invention provides crystalline granisetron base form I. The present invention further provides processes for preparing crystalline granisetron base form I. The granisetron base starting material can be obtained by any suitable method known in the art, including, e.g., by converting granisetron hydrochloride to the base in situ.

The present invention further provides a process for preparing crystalline granisetron base form I. In one embodiment, the present invention provides a process, which includes combining granisetron base (obtained, e.g., by in situ converting granisetron hydrochloride to granisetron base) with a solvent and heating to dissolve at least a portion of the granisetron base; allowing the solution to cool sufficiently to produce crystals of granisetron base form I; isolating the crystals, e.g., by filtration and, optionally, drying the crystals. In another embodiment, the present invention provides a process for preparing crystalline granisetron base form I, which includes combining granisetron base (obtained, e.g., by in situ converting granisetron hydrochloride to granisetron base) with a solvent and heating to dissolve at least a portion of the granisetron base; allowing the solution to cool sufficiently and adding an anti-solvent, optionally via gradual addition (e.g., drop-wise); mixing for sufficient period of time to produce crystals of granisetron base form I; isolating the crystals, e.g., by filtration and, optionally, drying the crystals.

The present invention further provides a process for preparing a salt of granisetron from crystalline granisetron base form I.

The processes of the present invention produce high purity crystalline granisetron base form I, which can be used as a convenient precursor for formulating pharmaceutical compositions that utilize granisetron base as the active agent or for preparing granisetron salts, e.g., granisetron hydrochloride, which are used in other types of dosage forms.

Granisetron base crystalline form I produces a unique X-ray powder diffraction pattern as depicted in FIG. 1 and Table 1, a unique infrared spectrum, which is depicted in FIG. 2, and also produces DSC and TGA curves as depicted in FIGS. 3 and 4, respectively.

### BRIEF DESCRIPTION OF THE DRAWINGS

FIG. 1 depicts the X-ray powder diffraction pattern of crystalline granisetron base form I.

FIG. 2 depicts the infrared spectrum of crystalline granisetron base form I.

FIG. 3 depicts the DSC curve of granisetron crystalline base form I.

FIG. 4 depicts the TGA curve of granisetron crystalline base form I.

### DETAILED DESCRIPTION OF THE INVENTION

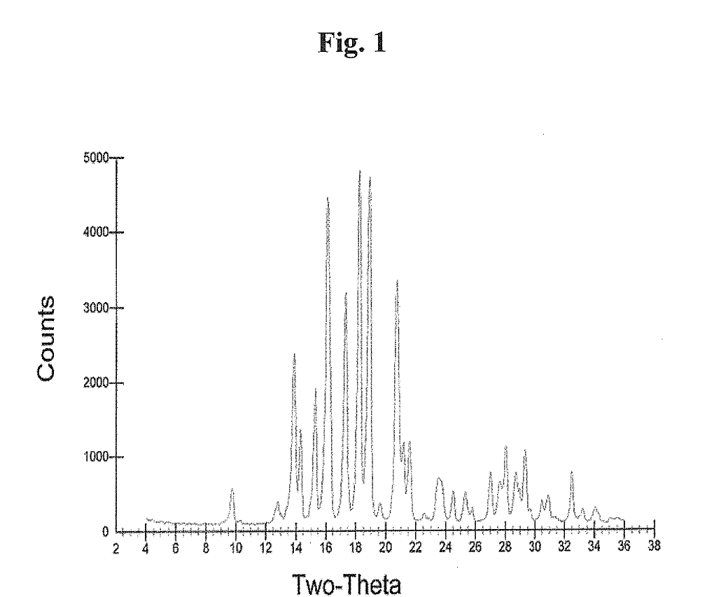

Granisetron base crystalline form I produces a unique X-ray powder diffraction pattern, which is different from the diffraction pattern of the crystalline granisetron base described in the '209 patent, as depicted in FIG. 1. The strong diffraction peaks at 14.0, 14.3, 15.3, 16.1, 17.3, 18.3, 19.0, 20.8, and 21.2±0.2 degrees 2θ are most characteristic of this form. The X-ray powder diffraction peak positions and intensities exhibited by granisetron base crystalline form I are listed in Table 1.

TABLE 1

| Peak position | Relative |
|---|---|
| 2θ degrees | intensity I/I₀ |
| 9.8 | 10.2 |
| 10.4 | 0.7 |
| 12.8 | 7.6 |
| 14.0 | 46.1 |
| 14.3 | 28.6 |
| 15.3 | 37.2 |
| 16.1 | 60.5 |
| 17.3 | 35.5 |
| 18.3 | 77.2 |
| 19.0 | 100.0 |
| 19.7 | 3.0 |
| 20.8 | 45.5 |
| 21.2 | 27.0 |
| 21.6 | 16.4 |
| 22.6 | 1.8 |
| 23.6 | 13.0 |
| 23.8 | 9.8 |
| 24.5 | 4.0 |
| 25.3 | 7.7 |
| 25.8 | 1.8 |
| 27.1 | 15.4 |
| 27.7 | 7.6 |
| 28.1 | 19.4 |
| 28.7 | 9.7 |
| 29.3 | 14.4 |
| 29.7 | 3.7 |
| 30.5 | 4.3 |
| 31.0 | 6.2 |
| 32.6 | 7.7 |
| 33.2 | 1.6 |
| 34.2 | 3.0 |
| 35.6 | 2.0 |

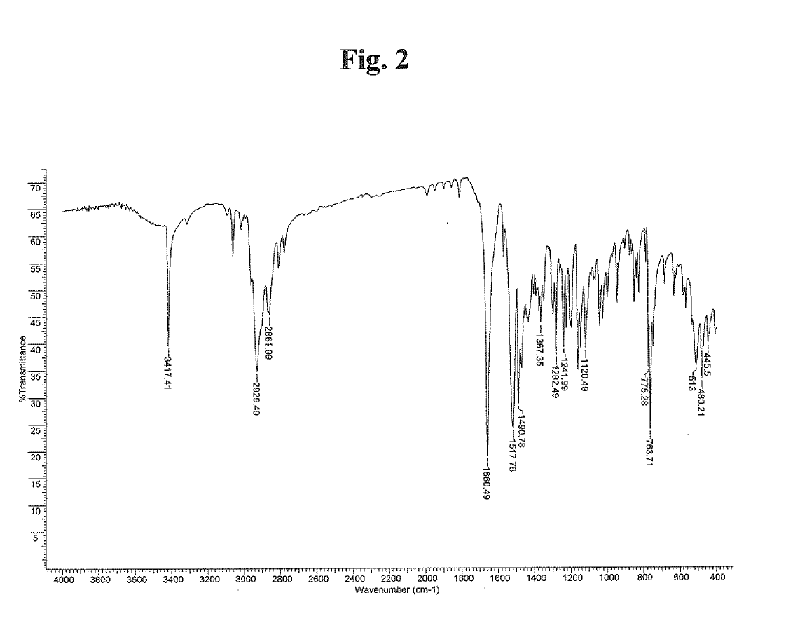

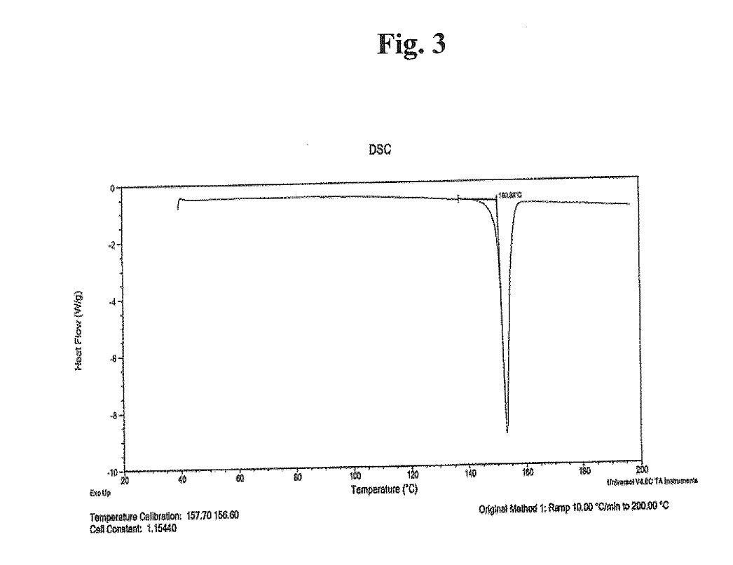

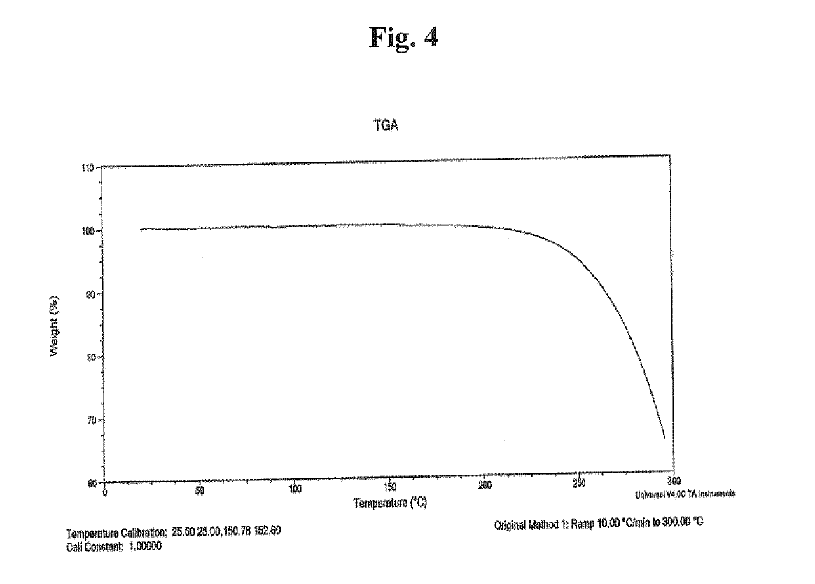

In addition, granisetron base crystalline form I produces a unique infrared spectrum as depicted in FIG. 2. Characteristic infrared spectrum absorption bands of the crystalline granisetron base form I appear at 3417, 2929, 2862, 1660, 1518, 1491, 1367, 1282, 1242, 1120, 775, 763, 513, 480, and 445±4 cm⁻¹. Further, granisetron base crystalline form I produces a characteristic DSC curve, exhibiting peak onset at 153±1° C. as depicted in FIG. 3, and a TGA curve as depicted in FIG. 4. The melting point of the granisetron base form I of the present invention is 152-154° C., which differs significantly from the melting point of 121-122° C. of the granisetron crystalline form described in the '209 patent.

In one embodiment, the present invention provides a process for preparing the crystalline granisetron base form I, which preferably includes:

combining granisetron base (obtained, e.g., by in situ converting granisetron hydrochloride to granisetron base) with a solvent and heating to dissolve at least a portion of the granisetron base;

allowing the solution to cool sufficiently, e.g., to produce crystals of granisetron base form I; and

isolating the crystals (e.g., collecting the crystals by filtration); and,

optionally drying the crystals.

Preferred solvents used for producing the granisetron base crystalline form I in accordance with the process of the present invention include cyclohexane, heptane, toluene, xylene, chloroform, acetone, methanol, ethanol, 1-propanol, 2-propanol, 1-octanol, benzyl alcohol, cyclohexanone, ethyl acetate, isobutyl acetate, tetrahydrofuran (THF), acetonitrile, dimethyl sulfoxide (DMSO), N,N-dimethylformamide (DMF), N,N-dimethylacetamide (DMA), ethylene glycol, and mixtures thereof.

The present invention also provides a process for preparing crystalline granisetron base form I, which preferably includes:

combining granisetron base (obtained, e.g., by in situ converting granisetron hydrochloride to granisetron base) with a solvent and heating to dissolve at least a portion of the granisetron base;

allowing the solution to cool and adding an anti-solvent, optionally via gradual addition (e.g., drop-wise);

mixing for sufficient period of time to produce crystals of granisetron base form I;

isolating the crystals, e.g., by filtration; and,

optionally, drying the crystals.

Preferred solvent systems for producing the granisetron base crystalline form I in accordance with the process of the present invention include solvent/anti-solvent combinations, which preferably include isobutyl acetate/methyl tert-butyl ether (MTBE), tetrahydrofuran (THF)/diisopropyl ether, methanol/water, isopropyl alcohol/water, acetone/water, acetonitrile/water, tetrahydrofuran (THF)/water, dimethyl sulfoxide (DMSO)/water, and chloroform/methyl tert-butyl ether (MTBE).

Preferably, the ratio between the granisetron base starting material and the crystallization solvent (granisetron base:solvent ratio) is at least about 0.5 g granisetron base per at least about 2.5 ml solvent.

When water is used as an anti-solvent, the water is preferably cold, e.g., cooled to a temperature below about 10° C., or cooled to a temperature below 5° C., prior to adding.

The present invention further provides a process for preparing a salt of granisetron from crystalline granisetron base form I. In one embodiment, the process includes reacting granisetron base (obtained, e.g., by dissolving at least a portion of crystalline granisetron base form I in a suitable solvent) with an acid (e.g., hydrochloric acid) to produce an acid addition salt of granisetron. An exemplary process includes reacting the granisetron base with hydrochloric acid to produce granisetron hydrochloride.

### EXAMPLES

The following examples further illustrate the invention but of course, should not be construed as in any way limiting its scope.

X-ray diffraction data were acquired using a PHILIPS X-ray diffractometer model PW1050-70. System description: K_{α1}=1.54178 Å, voltage 40 kV, current 28 mA, diversion slit=1°, receiving slit=0.2 mm, scattering slit=1° with a Graphite monochromator. Measurements of 2θ values typically are accurate to within ±0.2 degrees. Experiment parameters: pattern measured between 2θ=3° and 2θ=30° with 0.05° increments; count time was 0.5 second per increment.

Infrared spectra were run on Nicolet Fourier-transform infrared spectrometer model Avatar 360, with Omnic software version 5.2. All samples were run as KBr disks. The current infrared measurements are accurate to within 4 cm⁻¹.

Differential scanning calorimetry (DSC) measurements were run on TA instruments model Q1000, with Universal software version 3.88. Samples were analyzed inside crimped 40 μl Aluminum pans. Heating rate for all samples was 10° C./min

The crystalline granisetron base form I of the present invention was characterized by thermogravimetric analysis (TGA), a measure of the thermally induced weight loss of a material as a function of the applied temperature. Thermogravimetric analysis (TGA) was performed using a TA Instruments Q500 Thermal Analyzer with Universal Software (version 3.88). Samples were analyzed inside platinum baskets at heating rate of 5° C./minute.

### Example 1

This example demonstrates a method for producing crystalline granisetron base form I.

A reaction vessel was charged with 1.1 g of granisteron hydrochloride followed by addition of 20 ml water under mixing. Ammonium hydroxide (28%, 0.8 ml) was added and mixing was maintained for about 0.5 hour. Then, 20 ml of ethyl acetate was added and the layers were separated The organic layer was dried over magnesium sulfate and the solvent was evaporated to obtain a solid residue, which was delivered into a three-necked reaction vessel equipped with a thermometer, a reflux condenser and a mixer and mixed with 10 ml of acetone. The mixture was heated to reflux until a clear solution was obtained. The solution was cooled to room temperature and the resulting crystals were washed with 2 ml of cold acetone, filtered and dried to obtain granisetron base form I having 99.95% purity (by HPLC).

### Examples 2-6

These examples demonstrate processes for producing crystalline granisetron base form I.

A three-necked reaction vessel equipped with a thermometer, a reflux condenser and a mixer was charged with an initial weight of the free granisetron base, produced according to Example 1, and mixed with an organic solvent as indicated in Table 2. The organic solvents that were used and the volume of each solvent are summarized in Table 2. The mixture was heated to a certain temperature for each solvent, as detailed in Table 2, until a clear solution was obtained. The solution was cooled to room temperature and the resulting crystals were filtered, dried and weighed to obtain the final weight, as summarized in Table 2.

TABLE 2
Results with different solvents

| | | Volume | Temperature | Initial | Final | |
|---|---|---|---|---|---|---|
| Entry | Solvent | ml | ° C. | weight, g | weight, g | *Purity, % |
| 2 | ethyl acetate | 15 | reflux | 1.0 | 0.82 | 99.97 |
| 3 | isobutyl acetate | 10 | reflux | 1.3 | 0.73 | 99.95 |
| 4 | dimethyl sulfoxide | 2.5 | 80 | 1.0 | 0.70 | 99.98 |
| | (DMSO) | | | | | |
| 5 | 2-propanol:n-heptane | 10 | reflux | 1.18 | 0.75 | 99.99 |
| | (1:1) | | | | | |
| 6 | acetone:n-heptane | 20 | reflux | 1.75 | 1.06 | 99.98 |
| | (1:1) | | | | | |

*The purity was determined by HPLC.

### Example 7

This example demonstrates a process for producing crystalline granisetron base form I.

A three-necked reaction vessel equipped with a thermometer, a reflux condenser and a mixer was charged with 0.5 gram of granisetron free base and mixed with 5 ml of 2-propanol. The mixture was heated to reflux until a clear solution was obtained. Then, the solution was cooled to room temperature and cold water was added drop-wise until the solution became cloudy. The resulting crystals were filtered and dried to afford 0.33 g of granisetron base form I in 66% yield.

### Examples 8-11

These examples demonstrate processes for producing crystalline granisetron base form I from a mixture of a solvent and an anti-solvent.

A three-necked reaction vessel equipped with a thermometer, a reflux condenser and a mixer was charged with an initial weight of the free granisetron base, as described in Table 3, and mixed with an organic solvent. The organic solvents that were used and the volume of each solvent are summarized in Table 3. The mixture was heated to a certain temperature for each solvent, as detailed in Table 3, until a clear solution was obtained. Then, the solution was cooled to room temperature and an anti-solvent was added dropwise, as detailed in Table 3, until the solution became cloudy. The resulting crystals of granisetron base form I were filtered, dried and weighed to obtain the final weight, as summarized in Table 3.

TABLE 3
Results with different solvents and anti-solvents

| | | | Solvent | Initial | Final | | *Pu- |
|---|---|---|---|---|---|---|---|
| En- | | Anti- | volume | weight, | weight, | Temp. | rity |
| try | Solvent | solvent | ml | g | g | ° C. | % |
| 8 | isobutyl | MTBE | 10 | 1.0 | 0.55 | reflux | 99.98 |
| | acetate | | | | | | |
| 9 | methanol | water | 2 | 0.85 | 0.72 | reflux | 99.98 |
| 10 | acetone | water | 10 | 0.5 | 0.34 | reflux | 99.82 |
| 11 | DMSO | water | 3 | 0.6 | 0.6 | 80 | 99.68 |

*The purity was determined by HPLC.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Crystalline granisetron base form I characterized by an X-ray powder diffraction pattern exhibiting strong diffraction peaks at 14.0, 14.3, 15.3, 16.1, 17.3, 18.3, 19.0, 20.8, and 21.2±0.2 degrees 2θ.

2. The crystalline granisetron base form I of claim 1 further characterized by an infrared spectrum exhibiting characteristic absorption peaks at 3417, 2929, 2862, 1660, 1518, 1491, 1367, 1282, 1242, 1120, 775, 763, 513, 480, and 445±4 cm⁻¹.

3. The crystalline solid comprising granisetron base form I of claim 1 further characterized by DSC curve exhibiting peak onset at 153±1° C. and a melting point of from about 152° C. to about 154° C.

4. A process for preparing crystalline granisetron base form I, the process comprising:
combining granisetron base with a solvent and heating to dissolve at least a portion of the granisetron base;
cooling the solution, to produce crystals of granisetron base form I;
isolating the crystals (e.g., collecting the crystals by filtration); and,
optionally drying the crystals.

5. The process of claim 4, wherein the solvent comprises cyclohexane, heptane, toluene, xylene, chloroform, acetone, methanol, ethanol, 1-propanol, 2-propanol, 1-octanol, benzyl alcohol, cyclohexanone, ethyl acetate, isobutyl acetate, tetrahydrofuran (THF), acetonitrile, dimethyl sulfoxide (DMSO), N,N-dimethylformamide (DMF), N,N-dimethylacetamide (DMA), ethylene glycol, or a mixture thereof.

6. A process for preparing the crystalline granisetron base form I, the process comprising:
combining granisetron base with a solvent and heating to dissolve at least a portion of the granisetron base;
cooling the solution and adding an anti-solvent;
mixing to produce crystals of granisetron base form I;
isolating the crystals; and,
optionally drying the crystals.

7. The process of claim 6, wherein the solvent system comprises isobutyl acetate/methyl tert-butyl ether (MTBE), tetrahydrofuran (THF)/diisopropyl ether, methanol/water, isopropyl alcohol/water, acetone/water, acetonitrile/water, tetrahydrofuran (THF)/water, dimethyl sulfoxide (DMSO)/water, or chloroform/methyl tert-butyl ether (MTBE).

8. The process of claim 7, wherein the ratio between the granisetron base starting material and the crystallization solvent (granisetron base:solvent ratio) is at least about 0.5 g granisetron base per at least about 5 ml solvent.

9. The process of claim 7, wherein water is used as an anti-solvent, and the water is at a temperature below about 10° C. when introduced into the solvent mixture.

10. Granisetron base having a purity of at least about 98.5%.

11. Granisetron base having a purity of at least about 99.5%.

12. A process for preparing a salt of granisetron base, the process comprising converting crystalline granisetron base form I into a salt of granisetron.

13. The process of claim 12, wherein the granisetron base is reacted with an acid to produce an acid addition salt of granisetron.

14. The process of claim 13, wherein the acid is hydrochloric acid and the acid addition salt is granisetron hydrochloride.

15. A pharmaceutical composition comprising the granisetron base form I of claim 1 and pharmaceutically acceptable additives and excipients.
